(19) United States
(12) Patent Application Publication
(10) Pub. No.: US 2004/0209352 A1
(43) Pub. Date: Oct. 21, 2004

(54) Integrated electrode and cell immobilization device equipped with the integrated electrode

(76) Inventors: Nobuhiko Ozaki, Ikoma-shi (JP); Hiroaki Oka, Osaka (JP)

Correspondence Address: MCDERMOTT WILL & EMERY LLP, 600 13TH STREET, N.W., WASHINGTON, DC, 20005-3096, US

(21) Appl. No.: 10/694,396

(22) Filed: Oct. 28, 2003

(30) Foreign Application Priority Data
Oct. 28, 2002 (JP) 2002-313005

(51) Int. Cl. C12M 1/34

(52) U.S. Cl. 435/287.1

(57) ABSTRACT

An integrated electrode capable of immobilizing an isolated cell and/or a cultured cell on the electrode without giving any damage to its cell membrane, and capable of detecting an electrophysiological activity of the immobilized cell with sufficient sensitivity is provided. An integrated electrode having a substrate equipped with at least one electric conductor, and a wiring part which leads an electrical signal out from the aforementioned electric conductor, in which the integrated electrode can detect an electrical signal resulting from an electrophysiological change of a cell immobilized on the surface of the aforementioned electric conductor, and at least a part of the surface of the aforementioned electric conductor is coated with a dielectric material, with the aforementioned dielectric material being a positively charged polymer material, and the aforementioned cell being an isolated cell and/or a cultured cell.

### BACKGROUND OF THE INVENTION

1. Field of the Invention

The present invention relates to an integrated electrode for detecting an electrical signal resulting from an electrophysiological activity of an isolated cell and/or a cultured cell, and a cell immobilization device equipped with such an integrated electrode.

2. Description of the Related Art

Conventionally, techniques for detecting an electrophysiological activity of a cell have been known. Such techniques are used for e.g., screening of a drug utilizing an electrophysiological activity of a cell as a marker. An electrophysiological activity of a cell primarily refers to a degree of an activity of ion channels of a cell. In a cell, the ion concentration varies inside and outside of the cell membrane depending on the change of ionic permeability that corresponds to the activity of the ion channels. Therefore, detection of the degree of an activity of ion channels (electrophysiological activity of a cell) such as a mean open time, opening probability, frequency of openings and the like of ion channels is enabled by detecting the change of the electric potential within a cell.

As a method of detecting the change of the electric potential within a cell, a patch clamp method has been known. FIG. 17 is a cross sectional drawing schematically illustrating the principle of a patch clamp method. In a patch clamp method, as is shown in FIG. 17(a), a cell 102 is brought into close contact with the tip of a glass pipette 101 by, for example, aspirating the cell 102 with the glass pipette 101 first, and the cell membrane of the cell 102 within the area that is closely contacted to the glass pipette 101 is broken with additional force by aspiration to equilibrate the electric potential between inside of the pipette 101 and inside of the cell 102. Accordingly, the change of the electric potential within the cell 102 can be detected by detecting the electric potential of the electrode 103 within the pipette as the difference from the electric potential of the reference electrode 104 through the use of a detection means 104.

However, in the patch clamp method, the cell may be lead to death in approximately 1 hour because the cell membrane of the cell is broken. Further, the state of the cell of which cell membrane was broken is greatly different from the state in a living body. Therefore, it is difficult to obtain data which are useful for predicting electrophysiological activity of the cell in vivo. Moreover, the method requires skillful techniques for the operation in bringing a glass pipette into close contact with a cell, or the like, and takes a good amount of time to measure one sample. Thus, the method is not suitable for rapid screening of a large amount of candidate compounds as a drug.

For use in rapid screening of a drug, in particular, for use in the first screening (narrowing down of the first candidates), greater importance is placed on rapidness and simpleness of the measurement. Hence, an extracellular electric potential recording method in which a planar electrode is used is suitable for such a use (see, Japanese Patent No. 2949845, U.S. Pat. No. 5,810,725, U.S. Pat. No. 5,563,067, Japanese Laid-open Patent Publication No. 9-827318 and U.S. Pat. No. 5,187,069).

In the extracellular electric potential recording method in which a planar electrode is used, a biological sample such as a cell, a tissue piece or the like in a solution having the constitution which is approximate to the salt concentration in vivo is disposed on a planar electrode, and the change of electric potential of the electrode is measured to detect ion flow that passes through the ion channels. In other words, the extracellular electric potential recording method utilizes the event resulting from the electrophysiological activity of a cell to cause the change of the electric potential of an electrode that is disposed in the vicinity of the cell.

The extracellular electric potential recording method described above can execute the measurement by merely disposing a cell on a planar electrode, without any need of an operation such as bringing the cell into close contact with the glass pipette or the like, and the cell membrane of the cell is not destroyed. Therefore, an electrophysiological activity of a cell in a state which is approximate to its state in vivo can be rapidly measured in a convenient manner. Accordingly, the method is suited for rapid screening of a drug, and the like.

However, change of an electrical signal resulting from an electrophysiological activity of a cell is very weak, and the change becomes much weaker in the extracellular electric potential recording method in which a planar electrode is used, because the change of a signal has come to be detected via a solution. Therefore, for executing the measurement with higher accuracy, change of an electrical signal resulting from an electrophysiological activity of a cell is required to be detected with favorable sensitivity.

As a method for achieving the detection with favorable sensitivity, there exists a method of reducing impedance of a planar electrode. In a method of reducing impedance of a planar electrode, an electrode coated with platinum black has been suitably used. (see, Japanese Laid-open Patent Publication No. 6-78889).

Coating of an electrode with platinum black is accomplished by deposition of nano particles, i.e., aggregates of numerous platinum atoms yielded by electrolysis of a platinum salt or a platinum complex, on the surface of an electrode in a steric and complicated manner. Hence, the surface of an electrode becomes a rough face having an extremely great area because platinum black has a porous structure. Therefore, impedance of the electrode is reduced. Although the particle size of the aforementioned nano particles varies depending on the voltage which is applied upon the electrolytic plating, the diameter thereof is in the range of approximately 10 nm to 1 μm.

FIG. 18 is a scanning electron microscopy (SEM) photograph of the surface of platinum black deposited under a condition of the circuit resistance of 300 kΩ, and the applied voltage of 7 V. In FIG. 18, it is found that the diameter of the nano particles described above is in the range of approximately 60 nm to 160 nm. Moreover, in FIG. 18, it is revealed that nano particles are formed to give at least 15 layers in a vertical direction judging from the light and shade tone. Therefore, the thickness of the deposited platinum black is speculated to be approximately 0.9 μm (60 nm×15 layers) to 2.4 μm (160 nm×15 layers) from just within the area visible by the SEM photograph. Further, when viewed within the area of 1 μm², the difference of height between the highest protruding portion and the lowest depressed portion fall under the thickness of at least 5 layers of the nano particles. Therefore, the surface has the roughness of approximately 0.3 μm (60 nm×5 layers) to 0.8 μm (160 nm×5 layers). Hereinafter, the surface roughness herein is defined by the difference of height between the highest protruding portion and the lowest depressed portion within the area of 1 μm².

On the other hand, size of the cell to be immobilized on the electrode varies depending on the cell type, however, in the instance of HEK (Human Embryo Kidney) 293 cell, it has spherical shape having the diameter of approximately 10 to 15 μm when it is isolated, and the thickness thereof turns to be approximately 1 μm when it is settled on and attached to the substrate followed by extension. Accordingly, the surface roughness of an electrode coated with platinum black as shown in FIG. 18 is such roughness as not being negligible in respect of the thickness of the cell. Consequently, contact area between the surface of the electrode and the cell can not be sufficiently secured, and thus, it is difficult to immobilize a fine biological sample such as a cell or the like. Even with an electrode coated with platinum black, immobilization can be effected as long as the biological sample, such as a tissue piece, has a certain level of the thickness, i.e., the surface roughness of the electrode being negligible in respect of the thickness of the biological sample.

In order to immobilize an isolated cell on an electrode, it is required that the surface of the electrode has just such roughness which is negligible in respect of the thickness of the cell, and that it is nearly smooth. Specifically, surface roughness of the electrode is preferably 10% or less of the thickness of the isolated cell, for example, 0.1 μm or less. It may be theoretically possible to provide an electrode coated with platinum black having the surface roughness, which is approximate to the particle size of the nano particle that is an aggregate of platinum molecules, by adjusting the condition and the like of electrolysis. However, in such an instance, the effect of reducing impedance of the electrode is hardly achieved. Thus, the problem involving detection of a slight electrical signal derived from an isolated cell with favorable sensitivity can not be thereby solved.

Although a measuring device of the extracellular electric potential equipped with an electrode capable of readily immobilizing an isolated cell on its surface without destroying the cell membrane has been disclosed (see, pamphlet of International Publication No. WO02/055653), development of an electrode capable of measuring the extracellular electric potential with more favorable sensitivity has been desired.

### SUMMARY OF THE INVENTION

An object of the present invention is to provide an integrated electrode which can immobilize an isolated cell and/or a cultured cell on the electrode without degeneration, and which can detect an electrophysiological activity of the immobilized cell with sufficient sensitivity.

In order to accomplish the object as described above, the integrated electrode of the present invention comprises a substrate equipped with at least one electric conductor, and a wiring part which leads an electrical signal out from the aforementioned electric conductor, wherein the integrated electrode can detect an electrical signal resulting from an electrophysiological change of a cell immobilized on the surface of the aforementioned electric conductor, and wherein at least a part of the surface of the aforementioned electric conductor is coated with a dielectric material, with the aforementioned dielectric material being a positively charged polymer material, and the aforementioned cell being an isolated cell and/or a cultured cell.

Cells are negatively charged on their cell membrane in general, therefore, an electrostatic interaction is effected between the cell and a positively charged polymer material. Accordingly, on behalf of the surface of the electric conductor coated with a positively charged polymer material, the cell can be readily immobilized on the surface of the electric conductor, and it can be immobilized more firmly. The term “positively charged polymer” referred to herein is a polymer that is positively charged at the pH of the pKa value or less of the polymer. Since the dielectric material described above is responsible for immobilization of the cell via an electrostatic interaction that is exerted with the cell, degeneration of the cell is not caused. Also, as a matter of course, the cell is not thereby killed. The term “degeneration of a cell” referred to herein includes destruction of the cell membrane, chemical degeneration of the cell membrane, functional substances that are present in the cell membrane or on the cell membrane.

As the dielectric material described above, for example, a material involving a polymer material selected from the group consisting of polyethyleneimine, polyornithine, and polylysine can be used. Alternatively, a material involving a polymer material having a biguanide group or a carbamoylguanide group can be used.

The aforementioned electric conductor is preferably produced with a material selected from the group consisting of platinum, gold, palladium, rhodium, silver, tungsten, ITO, and any mixtures thereof.

Furthermore, in the integrated electrode described above, at least a part of the surface of the aforementioned electric conductor that is coated with the aforementioned dielectric material maybe further coated with an immobilization material. In this instance, the aforementioned immobilization material is a material that provides an electrostatic interaction and/or intermolecular force with the aforementioned cell, and that is different from the aforementioned dielectric material. By the coating with the aforementioned dielectric material, and additional coating with the aforementioned immobilization material, immobilization of the cell to the electric conductor can be more facilitated and/or more strengthened. Furthermore, the aforementioned immobilization material is responsible for immobilization of the cell by the electrostatic interaction and/or intermolecular force that is provided with the aforementioned cell, therefore, the cell is not thereby killed, and the cell is not degenerated, similarly to the effect achieved by the aforementioned dielectric material. As the aforementioned immobilization material, for example, a protein having adhesiveness with a cell can be used.

As the capacitance of the electric double layer of the interface between the aforementioned electric conductor and a solution becomes larger, the effect of reducing impedance of the electric conductor becomes greater, which is preferred. Specifically, it is preferred that the capacitance of the electric double layer of the interface with a 0.1 M electrolyte solution of the aforementioned electric conductor becomes 27 μF/cm² or greater at the applied voltage of 0 V, in light of the detection sensitivity of the electrical signal resulting from the aforementioned electrophysiological activity of a cell.

Additionally, the aforementioned integrated electrode may be constituted such that the aforementioned electric conductor is formed within at least one through-hole formed to the substrate and/or around the aforementioned through-hole on the upper face of the aforementioned substrate. In this instance, the cell can be captured in the aforementioned through-hole, or the cell can be aspirated from beneath the aforementioned through-hole, therefore, immobilization of the cell can be more facilitated. It may be constituted such that one electric conductor is formed per one through-hole described above, alternatively, one electric conductor is formed per a set of multiple through-holes described above.

The cell immobilization device of the present invention is equipped with the aforementioned integrated electrode, and a solution retaining part for culturing the aforementioned cell in a region including the surface of the aforementioned electric conductor. When multiple electric conductors are formed on the aforementioned substrate of the aforementioned integrated electrode, the aforementioned solution retaining part may be separated per each one electric conductor, or may be separated per a set of the multiple electric conductors.

The object as described above, other objects, characteristics, and advantages of the present invention will be apparent from the following detailed description of the preferred embodiments with reference to the accompanying drawings.

### BRIEF DESCRIPTION OF THE DRAWINGS

FIG. 1 is a cross sectional drawing schematically showing the constitution of the extracellular electric potential measuring apparatus according to the first Embodiment.

FIG. 2 is a cross sectional drawing viewed along the line A-A depicted in FIG. 1, schematically showing the constitution of the extracellular electric potential measuring apparatus according to the first Embodiment.

FIG. 3 is a schematic drawing showing the extracellular electric potential measuring apparatus according to the first Embodiment.

FIG. 4 is a top view showing the sensor part of the cell immobilization device according to the second Embodiment.

FIG. 5 is a cross sectional drawing schematically showing the constitution of the cell immobilization device according to the third Embodiment.

FIG. 6 is a cross sectional drawing viewed along the line B-B depicted in FIG. 5, schematically showing the constitution of the cell immobilization device according to the third Embodiment.

FIG. 7 is a top view showing the sensor part of the cell immobilization device according to the fourth Embodiment.

FIG. 8 is a cross sectional drawing schematically showing the constitution of the cell immobilization device according to the fifth Embodiment.

FIG. 9 is a cross sectional drawing schematically showing the constitution of the cell immobilization device according to the sixth Embodiment.

FIG. 10 is a cross sectional drawing viewed along the line C-C depicted in FIG. 9, schematically showing the constitution of the cell immobilization device according to the sixth Embodiment.

FIG. 11 is a top view showing the sensor part of the cell immobilization device according to the seventh Embodiment.

FIG. 12 is a drawing illustrating the measurement results of the impedance in Example 1 and Comparative Example 1 for the applied frequency.

FIG. 13 is a drawing illustrating the measurement results (voltage vs. elapsed time) in Example 2.

FIG. 14 is a drawing illustrating the measurement results (voltage vs. elapsed time) in Comparative Example 2.

FIG. 15 is a drawing showing the stereoscopic microscopy photograph in Example 3.

FIG. 16 is a drawing showing the stereoscopic microscopy photograph in Comparative Example 3.

FIG. 17 is a cross sectional drawing schematically illustrating the principle of a patch clamp method.

FIG. 18 is a SEM photograph of the surface of platinum black deposited by electrolysis.

### DETAILED DESCRIPTION OF THE INVENTION

The present invention is explained in more detail with reference to the drawings.

### First Embodiment

[Constitution of Cell Immobilization Device]

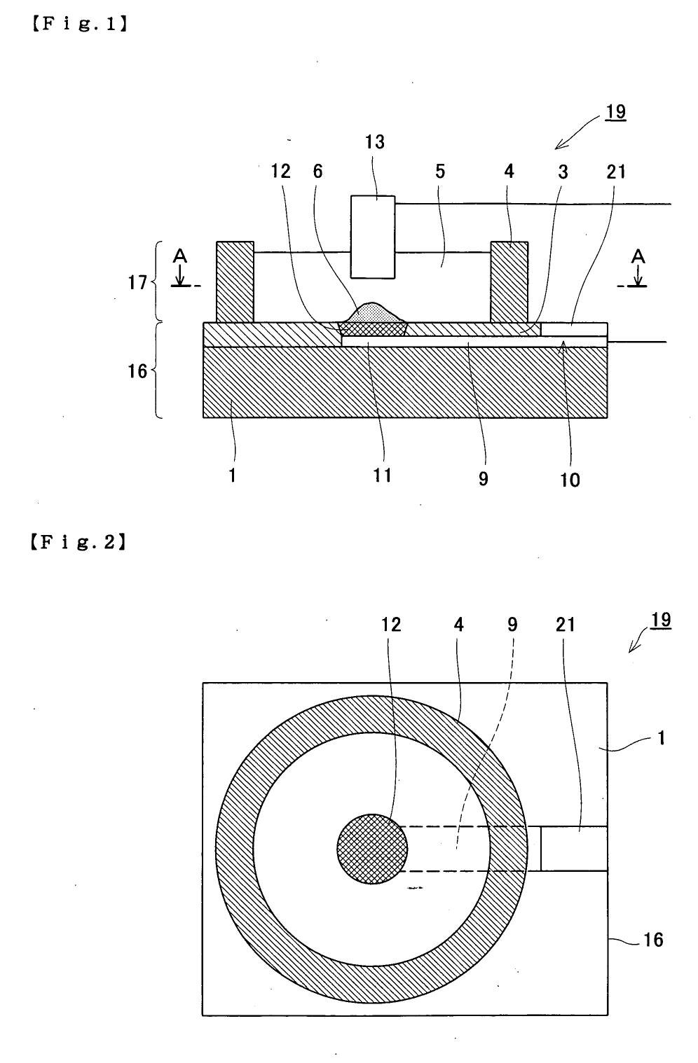

This Embodiment relates to an extracellular electric potential measuring apparatus equipped with a cell immobilization device for detecting an electrical signal resulting from an electrophysiological change of a cell. FIG. 1 is a cross sectional drawing schematically showing the constitution of a cell immobilization device 19 that constitutes a part of the extracellular electric potential measuring apparatus according to this Embodiment. FIG. 2 is a cross sectional drawing viewed along the line A-A depicted in FIG. 1. Although FIG. 1 illustrates a state in which a cell 6 is anchored on the cell immobilization device 19, the cell 6 is omitted in FIG. 2. Moreover, in FIG. 2, a lead wire 9 formed on the back face of a sensor part 16 is illustrated by a dashed line.

The cell immobilization device 19 comprises a sensor part 16 (corresponding to “integrated electrode” referred to herein) equipped with an electrode (corresponding to “electric conductor” referred to herein) 11, and a solution retaining part 17. The sensor part 16 comprises an electrode 11 and a substrate 1 equipped with a lead wire 9 that connects to the electrode 11. The upper face of the electrode 11 is coated with a dielectric layer 12, and the lead wire 9 is coated with an insulating layer 3 on its upper face except for the external connection part 10. The upper face of the external connection part 10 of the lead wire 9 is coated with a coating layer 21. For the coating layer 21, an electric conductive material that has potent resistance to the surrounding atmosphere is selected depending on such a surrounding atmosphere to which the external connection part 10 is exposed. Durability of the external connection part 10 is improved by coating with the coating layer 21, however, it may not be necessarily coated by the coating layer 21.

Top face shape of the electrode 11 is preferably circular or square, e.g., with the diameter or length of one side in the range of approximately 1 μm to 2000 μm. When size of the electrode is greater than the subject cell to be measured, an electrical signal derived from an electrophysiological activity of multiple cells can be detected with one electrode.

In a sensor part 16 for measuring an electrophysiological activity of a single isolated cell, for example, when the subject cell to be measured has a major axis of approximately 15 μm, the electrode 11 may be circularly shaped having the diameter of approximately 5 μm, although it may vary depending on the subject cell to be measured. In this instance, one isolated cell can be readily immobilized on the electrode 11, and thus, an electrical signal resulting from electrophysiological change of the one isolated cell can be readily detected.

The cell immobilization device 19 is equipped with a solution retaining part 17 on the sensor part 16, for use in culturing the cell. The solution retaining part 17 comprises a cylindrical divider member 4 provided on the sensor part 16, an inner region of the divider member 4, and a reference electrode 13 provided within the inner region.

The reference electrode 13 has only to be immersed in a cell culture medium 5 during the measurement, and it may be previously fixed within the solution retaining part 17, or may be placed and fixed in the culture medium 5 upon the measurement. For example, the reference electrode 13 may be mounted on the inside wall of the divider member 4, although not shown in the Figure.

Examples of the substrate which is preferably used as the substrate 1 include those formed with a semiconductor material typified by single crystal silicon, amorphous silicon, silicon carbide, silicon dioxide, silicon nitride and the like; a composite material of these semiconductor materials typified by a silicon.on.insulator (SOI) and the like; an inorganic insulating material selected from the group consisting of glass, quartz glass, alumina, sapphire, forsterite, silicon carbide, silicon dioxide, and silicon nitride; and an organic material selected from the group consisting of polyethylene, ethylene, polypropylene, polyisobutylene, polyethylene terephthalate (PET), unsaturated polyester, fluorocarbon resin, polyvinyl chloride, polychlorinated vinylidene, polyvinyl acetate, polyvinyl alcohol, polyvinyl acetal, an acrylic resin, polyacrylonitrile, polystyrene, an acetal resin, polycarbonate (PC), polyamide, a phenol resin, a urea resin, an epoxy resin, a melamine resin, a styrene.acrylonitrile copolymer, an acrylonitrile.butadiene styrene copolymer, a silicon resin, polyphenylene oxide and polysulfone. More preferably, a substrate that is formed with single crystal silicon, SOI, PET, or PC may be used.

The electrode material which is preferably used for forming an electrode 11 may be a metal material selected from the group consisting of platinum, gold, palladium, rhodium, silver, and tungsten; or a metal oxide material selected from the group consisting of titanium oxide, tin oxide, manganese oxide, lead oxide, and indium tin oxide (ITO). The electrode 11 may be formed using one material selected from these materials, or multiple kinds of materials may be deposited into e.g., layers, to form the electrode 11. Smoothness of the upper face of the electrode 11 is preferably secured, with the surface roughness of 0.1 μm or less, and such smoothness may be secured by coating with an electric conductive polymer or a unimolecular membrane. Also as a lead wire material which forms the lead wire 9 which is connected to the electrode 11, similar materials to those for the aforementioned electrode material can be used.

As the dielectric material for forming the dielectric layer 12, a positively charged polymer material such as polyethyleneimine (PEI), polyornithine (PO), polylysine (PL) or the like is suitably used. The dielectric layer 12 has an action of lowering the impedance of the electrode 11, accompanied by the action of attracting a cell having a positively charged surface by an electrostatic interaction through being formed with a positively charged polymer, as demonstrated in Example 3 below. Because sialic acid is present on the surface of a cell membrane, the sialic acid is responsible for a negative charge of the cell surface. In the cell immobilization device of this Embodiment, because the cell is immobilized on the upper face of the electrode utilizing the aforementioned electrostatic interaction, destruction of the cell membrane, chemical degeneration of the cell membrane, and functional substances that are present in the cell membrane or on the cell membrane and the like are not caused. Therefore, electrophysiological change of the cell in its living state, and in a state which is approximate to its state in a living body can be detected. Either an isolated cell or a cultured cell can be used as the subject cell to be measured.

Moreover, a polymer material having other strongly basic functional group is suitably used as the dielectric material. The strongly basic functional group in the polymer is positively charged in a condition where it is exposed to the cell culture medium, therefore, an electrostatic interaction is raised with a cell having a negatively charged surface. For example, a polymer material having a strongly basic functional group such as a biguanide group, a carbamoylguanidide group or the like is suitably used. Specific examples of such polymer include allylbiguanide-co-allylamine (PAB), allyl-N-carbamoylguanidino-co-allylamine (PAC) and the like.

The dielectric layer 12 can be formed by exposing a dielectric material including a dielectric material dissolved at predetermined concentration onto an electrode 11, removing the dielectric material solution from the upper face of the electrode 11 after elapse of a predetermined time period, washing the upper face at least once with a washing liquid, and drying. Alternatively, a method in which the aforementioned dielectric material solution is spotted only onto the upper face of an electrode 11 to execute the coating is also acceptable.

Coating of the electrode with the dielectric layer 12 does not affect the smoothness of the surface of the electrode 11 in respect of the size of the cell. PEI has molecular weight of 600 to 750000, and has the structural formula (I) shown below.

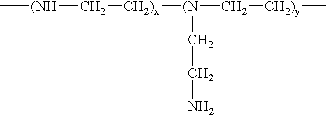

In the structural formula (I) described above, x and y are defined as: 5≦x≦9000 and 5≦y≦9000.

For example, when PEI is used as the dielectric material 12, thickness of the molecule predicted from the interatomic distance of each functional group is about 5 nm. PEI is speculated as having just the thickness and roughness in the approximate order which can be derived from the molecular structure. Although data not shown, when scanning electron microscopy (SEM) photographs are observed for the substrate surface not coated with PEI and the substrate surface coated with PEI, almost no difference is found between both cases. Such an observation also suggests that surface roughness of the electrode 11 coated with PEI is almost the same level with surface roughness of the electrode 11 not coated therewith. More specifically, when surface roughness of the electrode 11 is equal to or less than 0.1 μm, surface roughness of the electrode 11 coated with PEI also becomes approximately 0.1 μm or less. Accordingly, the surface of the electrode 11 coated with PEI is smooth, which is beyond comparison with platinum black shown in FIG. 18, and surface roughness thereof is negligible in respect of the thickness of the cell, and thus it is not an obstacle in immobilizing the cell on the upper face of the electrode. The fact that coating with PEI is not an obstacle in immobilizing a cell is also ascertained in Example 3 below.

As is represented by the above structural formula, PEI has a NH₂ group which is a strongly basic functional group that is positively charged in a cell culture medium, therefore, an electrostatic interaction is effected with the cell which is negatively charged.

Although impedance of the electrode varies depending on the coating conditions and the like of the dielectric material, impedance of the electrode is reduced by coating the electrode with the dielectric material. As exemplary impedance which is responsible for the impedance of an electrode, there exists the impedance owing to the capacitance of the electric double layer of the interface with the solution of the electrode. The coating of the electrode is carried out such that the capacitance of the electric double layer becomes preferably equal to or greater than 25 μF/cm², more preferably equal to or greater than 27 μF/cm², with the applied voltage of 0 V, and the electrolyte concentration of 0.1 M.

Examples of the material of the insulating layer 3 for coating and insulating the lead wire 9 include resins such as a polyimide (PI) resin, an epoxy resin and the like. Preferably, a photosensitive resin such as negative photosensitive polyimide (NPI) is used. In instances where a photosensitive resin is used, the insulating layer 3 can be formed in an arbitrary region by applying the photosensitive resin on the entire upper face of the sensor part 16 first, and thereafter removing the insulating layer 3 which was formed on the electrode 11 and on the external connection part 10 of the lead wire 9 utilizing pattern formation by photoetching to result in exposure of the electrode 11 and external connection part 10. It is preferred that the insulating layer 3 is formed by the method as described above in light of the production efficiency.

The electrical signal from the electrode 11 is measured with the electric potential of the reference electrode 13 as a standard. In general, the reference electrode 13 preferably has its surface area of equal to or greater than the surface area of the electrode 11, and is made from a material such as gold, platinum, silver-silver chloride or the like, however, size and shape thereof can be determined ad libitum.

The divider member 4 can be made from for example, acryl. The divider member 4 is acceptable as long as it is constituted such that it can retain a cell culture medium 5 in its inner region of which bottom face agrees with the upper face of the sensor part 16 including the upper face of the electrode 11. Thus, but the shape of the divider member is not limited to cylindrical.

[Method of Producing Cell Immobilization Device]

An example of the method of producing a cell immobilization device 19 is illustrated. A desired pattern is formed having multiple sets, the set being an electrode 11 and a corresponding lead wire 9 as one set, by vapor deposition of an electrode material on a substrate 1 first, followed by use of photoresist to execute the etching. Thereafter, the upper face of the lead wire 9 except for an external connection part 10 is coated with an insulating layer 3. Then, the upper face of the electrode 11 is coated with a dielectric material to form a dielectric layer 12. Further, the upper face of the external connection part 10 of the lead wire 9 is coated with a coating layer 21. Thereafter, the substrate 1 is cut out into small pieces with a predetermined angle. Thus, one small piece is defined as a sensor part 16. One small piece is produced such that one set of the electrode 11 and the lead wire 9 are formed therein. Pattern of the electrode 11 may be formed by a lift off method or a masking method in which vapor deposition is conducted through a stencil mask having the aforementioned pattern previously formed. On upper face of thus formed sensor part 16 is adhered a divider member 4 to form a solution retaining part 17.

[Method of Using Cell Immobilization Device]

First, a subject cell 6 to be measured is immobilized on the upper face of the electrode 11. An immobilization material is applied, which is different from the dielectric material that forms the dielectric layer 12, to the region within the divider member 4 of the uppermost face of the sensor part 16, on at least the upper face of the electrode 11. As the immobilization material, a material that exerts an electrostatic interaction and/or intermolecular force with a cell 6 is used. By coating the upper face of the electrode 11 with such a material, immobilization of the cell 6 on the electrode 11 can be facilitated and/or strengthened.

The dielectric layer 12 which was coated on the upper face of the electrode 11 has an action to attract a cell via an electrostatic interaction, therefore, a constitution to permit the immobilization of a cell 6 on the upper face of the electrode 11 can be achieved even though an immobilization material is not applied, on behalf of such an action. However, immobilization of a cell 6 can be more facilitated and/or strengthened by additionally coating the upper face of the electrode 11 with an immobilization material. Coating of the upper face of the electrode 11 with the immobilization material may be conducted prior to setting of the divider member 4.

The immobilization material which may be used is a material which does not degenerate the cell membrane of a cell. Therefore, any material that effects immobilization of a cell through the occurrence of a crosslinking reaction between the immobilization material and a cell membrane is not used for the purpose of detecting an electrical signal derived from an electrophysiological change of a cell which is approximate to a state in a living body. The immobilization material which may be used is for example, a matrix material. The matrix material which may be suitably used is a cell adhesive protein. Examples of such a protein include collagen, fibronectin, vitronectin, laminin and the like. Coating of the upper face of the electrode 11 with an immobilization material may not be necessarily conducted.

Next, a cell culture medium 5 is charged to fill in the solution retaining part 17. Examples of the cell culture medium 5 which may be suitably used include physiological saline solutions containing 20 mM or greater and 400 mM or less sodium chloride as a principal component; and media containing any of a variety of nutrients, growth factors, antibiotics and the like; buffer solutions including a predetermined chemical substance, compound, drug or the like dissolved therein.

Then, a desired cell 6 is seeded into the cell culture medium 5. Concomitantly to the progress of culture of the cell 6, the adhesive cell is immobilized on the uppermost face of the sensor part 16.

Measurement of an electrical signal resulting from an electrophysiological change of a cell is initiated in a state in which the cell 6 is immobilized on the uppermost face of the sensor part 16. Measurement of the electrical signal refers to the measurement of the difference of electric potential between the electrode 11 and the reference electrode 13, on the basis of the electrical signals detected from a pair of electrodes 11 and 13. In a cell, as described above, ion permeability of the cell membrane is changed corresponding to the change of the activity of its ion channels, and the ion concentration inside and outside of the cell membrane is changed accompanied by such a change of ion permeability. In other words, gradient of the ion concentration inside and outside of the cell membrane is changed. Accompanying to such change of the gradient of the ion concentration, the difference of electric potential between the electrode 11 and the reference electrode 13 is changed. Therefore, by measuring the aforementioned electric potential difference, an electrophysiological change of the cell can be indirectly detected. The aforementioned electric potential difference can be measured by for example, the extracellular electric potential measuring apparatus described below.

[Constitution of Extracellular Electric Potential Measuring Apparatus]

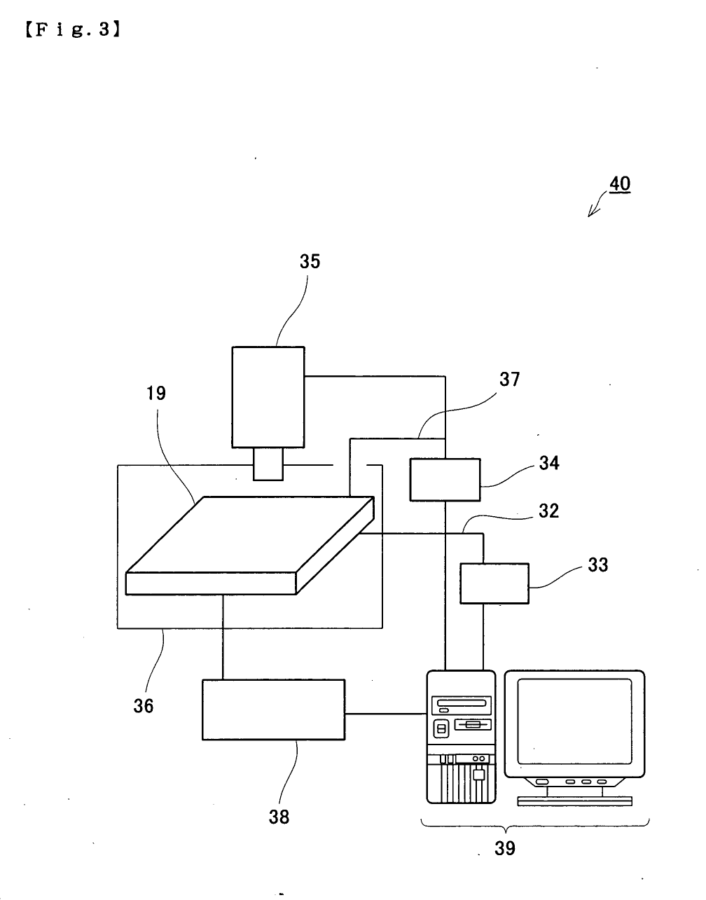

FIG. 3 is a schematic drawing showing the extracellular electric potential measuring apparatus according to this Embodiment. The extracellular electric potential measuring apparatus 40 comprises a controlling part 39, a signal amplification part 33 connected thereto, a stimulatory signal imparting part 34 and a solution driving part 38, an imaging part 35, and a mounting part 36.

A cell immobilization device 19 is mounted to the mounting part 36. The mounting part 36 has functions to keep the mounted cell immobilization device 19 at a predetermined temperature, gas concentration and humidity. The controlling part 39 detects and records the electric potential difference between the electrodes 11 and 13 of the cell immobilization device 19, on the basis of the signal entered from the signal amplification part 33. In addition, the controlling part 39 controls the stimulatory signal imparting part 34 on the basis of the predetermined stimulating condition. The stimulatory signal imparting part 34 is equipped with a D/A transducer, and electrical stimulation is applied to the cell on the cell immobilization device 19 via the transducer and a line 37. The electrical signal from the cell immobilization device 19 is lead out to a signal amplification device 33 via the line 32. In the signal amplification device 33, the electrical signal is amplified, subjected to the limitation of the frequency band, and entered into the controlling part 39 via the A/D transducer.

The solution driving part 38 has functions to discharge the culture medium 5 retained within the solution retaining part 17 of the cell immobilization device 19, or to inject the culture medium 5 into the solution retaining part 17, and is driven by the controlling part 39 as needed. Using the imaging part 35, the electrode 11 on the cell immobilization device 19 can be put into an image or observed. Further, the stimulatory signal imparting part 34 may be constituted such that the output stimulatory signal is selected, on the basis of imaging data from the imaging part 35.

The extracellular electric potential measuring apparatus 40 imparts a stimulatory signal from the stimulatory signal imparting part 34 to the cell 6, and can detect the electrophysiological change of the cell 6 corresponding thereto. Alternatively, it is also possible to detect the electrophysiological change which is spontaneously generated in the cell, without imparting a stimulatory signal.

### Second Embodiment

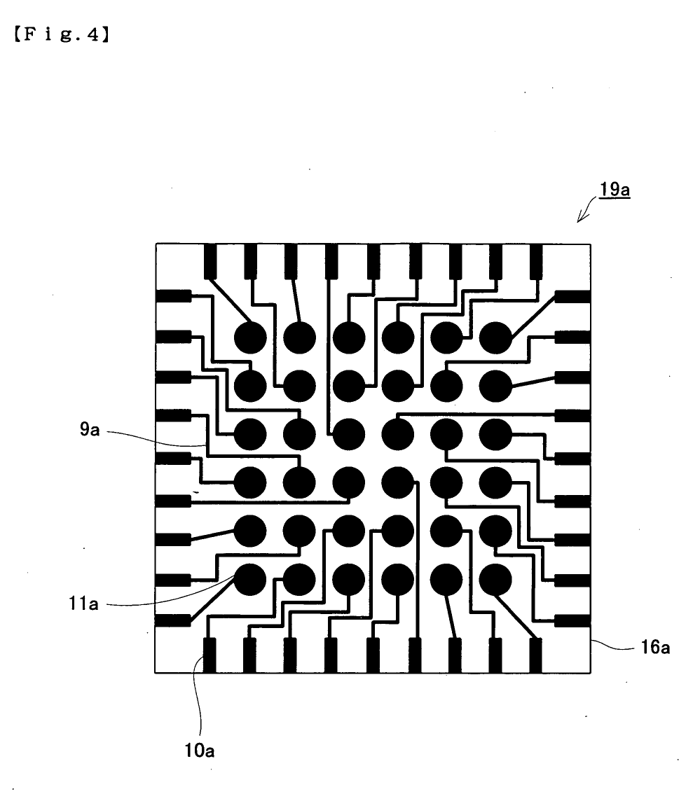

FIG. 4 is a top view showing the sensor part 16a of the cell immobilization device 19a according to the second Embodiment. The cell immobilization device 19a is constituted such that electrodes 11a are arranged at each intersecting point of a lattice having 6 rows and 6 columns. The cell immobilization device 19 of the first Embodiment has the constitution having only one electrode 11 formed on the sensor part 16, however, the cell immobilization device 19a of this Embodiment is constituted to have multiple electrodes 11a and corresponding lead wires 9a formed on the sensor part 16a.

Although FIG. 4 illustrates with a solution retaining part omitted therefrom, the divider member (having a similar constitution to the divider member 4 of the first Embodiment) that constitutes the solution retaining part may be provided either per every one electrode 11a, or per a set of multiple electrodes 11a. The constitution provided with the divider member per every one electrode 11a is useful, for example, in the measurement of responsiveness to a drug of the immobilized cell on each electrode 11a, whilst the constitution provided with the divider member per a set of multiple electrodes 11a is useful, for example, in carrying out the analysis of a network because a network can be formed among nerve cells immobilized on each electrode 11a.

Other constitutions, methods of use and the like of the cell immobilization device 19a are similar to those of the cell immobilization device 19 of the first Embodiment, therefore, the explanation thereof is now omitted.

### Third Embodiment

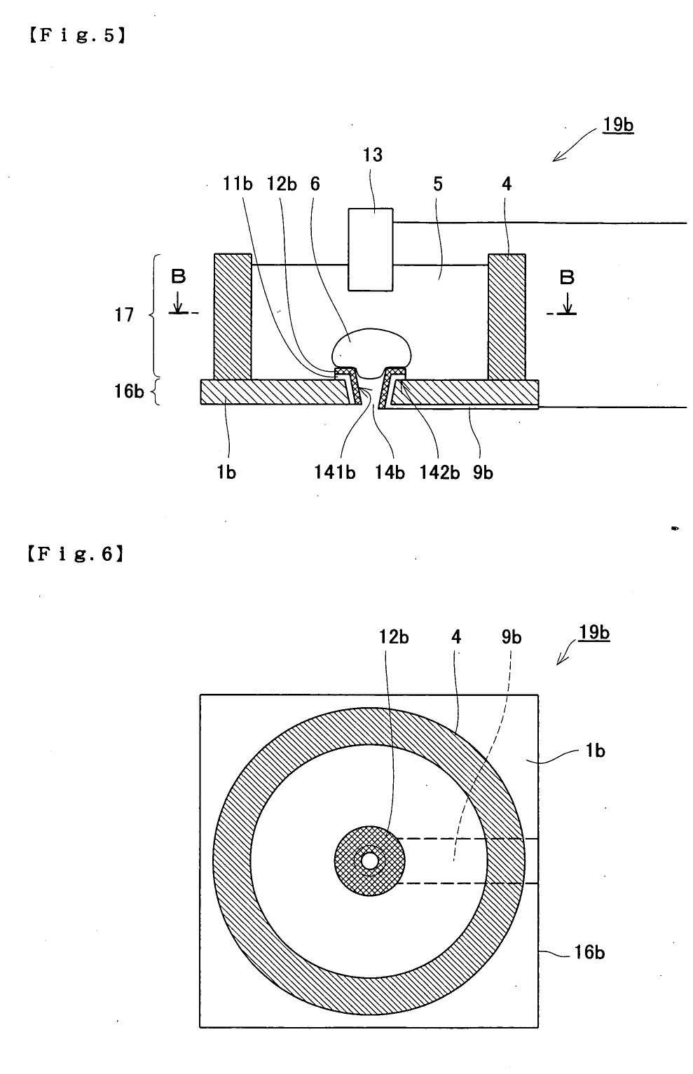

FIG. 5 is a cross sectional drawing schematically showing the constitution of the cell immobilization device 19b according to this Embodiment. FIG. 6 is a cross sectional drawing viewed along the line B-B depicted in FIG. 5. However, FIG. 5 illustrates the state in which a cell 6 is immobilized in the cell immobilization device 19, but FIG. 6 illustrates with the cell 6 omitted therefrom. Moreover, in FIG. 6, a lead wire 9 formed on the lower face of the sensor part 16 is illustrated by a dashed line.

Because the cell immobilization device 19b of this Embodiment has a different constitution in only the sensor part from the cell immobilization device 19 of the first Embodiment, the explanation of other constitutions except for the sensor part is omitted through assigning the identical number.

A substrate 1b has a through-hole 14b. An electrode 11b is formed on the hole wall surface 141b and the marginal edge of the hole opening 142b of the through-hole 14b. The electrode 11b is formed by making an electrode material adhered on the hole wall surface 141b and the marginal edge of the opening 142b of the through-hole 14 using a vacuum vapor deposition method or a sputtering method. The surface of the electrode 11b is coated with the dielectric layer 12b. For coating of the electrode 11b with the dielectric layer 12, a similar method to that in the first Embodiment can be employed. Coating of the electrode 11b with a dielectric layer 12b may be carried out such that the capacitance of the electric double layer of the interface of the electrode 11b with a 0.1 M electrolyte solution becomes preferably equal to or greater than 25 μF/cm², more preferably equal to or greater than 27 μF/cm², with the applied voltage of 0 V, similarly to the first Embodiment.

On the lower face of the sensor part 16b is formed a lead wire 9b such that it connects to the electrode 11b. The lead wire 9b may be constituted such that it is formed on the upper face of the sensor part 16b.

The through-hole 14b has a truncated cone shape having the upper opening greater than the lower opening. A part of a cell 6 is captured by the through-hole 14b, and the cell 6 is retained on the sensor part 16b with close contact thereto. Since the through-hole 14b has a truncated cone shape, wide contact area with the cell 6 can be secured. However, the shape of the through-hole 14b is not limited to such a truncated cone shape (for example, see, the fifth Embodiment), but any shape capable of capturing a part of a cell 6 is acceptable. Size of the through-hole 14b which can be employed may be an arbitrary size depending on the subject cell 6 to be captured. For example, the diameter of the opening on the upper face of the sensor part 16b is in the range of 10 μm or greater and 500 μm or less, and usually, the diameter is in the range of 10 μm or greater and 100 μm or less. Suitably, an illustrative example may be that the diameter of the opening is approximately 20 μm, when the major axis of the cell which is used as a biological sample is approximately 30 μm.

Method of forming a through-hole 14b may vary depending on the material of the substrate 1b, however, when the substrate 1b consists of PET, for example, it can be formed using an excimer laser. In addition, when the substrate 1b is a Si wafer, for example, it can be formed by etching.

Moreover, a constitution is permitted in which an aspiration means is provided capable of aspirating the cell 6 from beneath the through-hole 14b. Such a constitution enables capturing of the cell 6 on the through-hole 14b to be more strengthened, and thus the cell can be captured on the through-hole 14b even though the cell tends to float.

Any of the substrate material for forming the substrate 1b, the electrode material for forming the electrode 11b, and the dielectric material for forming the dielectric layer 12b which can be used may be the material as presented in the first Embodiment. Method of using the cell immobilization device 19b is identical to the method of using the cell immobilization device 19 as in the first Embodiment.

### Fourth Embodiment

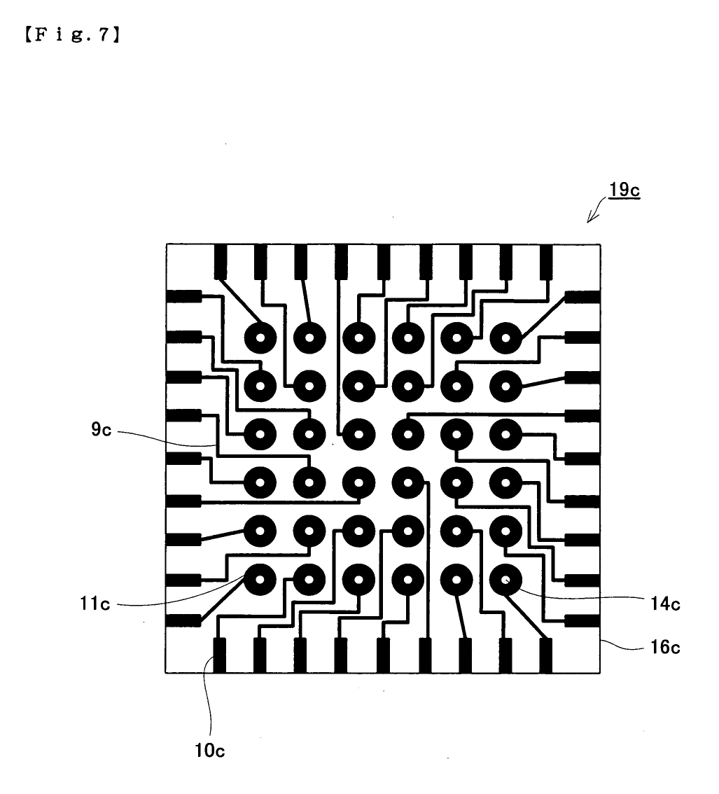

FIG. 7 is a top view showing the sensor part 16c of the cell immobilization device 19c according to the fourth Embodiment. A lead wire 9c is not appeared on the upper face because it is formed on the lower face of the sensor part 16c, however, in FIG. 7, the lead wire 9c is shown in the upper face for the sake of convenience. The cell immobilization device 19c is constituted such that electrodes 11c are arranged at each intersecting point of a lattice having 6 rows and 6 columns. The cell immobilization device 19b of the third Embodiment has the constitution including only one electrode 11b formed on the sensor part 16b, however, the cell immobilization device 19c of this Embodiment is constituted to have multiple electrodes 11c and corresponding lead wires 9c formed on the sensor part 16c.

Although FIG. 7 illustrates with a solution retaining part omitted therefrom, the divider member (having a similar constitution to the divider member 4 of the third Embodiment) that constitutes the solution retaining part may be provided either per every one electrode 11c, or per a set of multiple electrodes 11c. The constitution provided with the divider member per every one electrode 11c is useful, for example, in the measurement of responsiveness to a drug of the immobilized cell on each electrode 11c, whilst the constitution provided with the divider member per a set of multiple electrodes 11c is useful, for example, in carrying out the analysis of a network because a network can be formed among nerve cells immobilized on each electrode 11c. A lead wire 9c is not formed on the upper face of the sensor part 16c, therefore, the sensor part 16 and the divider member are not necessarily constituted separately, but it is possible to give an integrated formation.

Other constitutions, methods of use and the like of the cell immobilization device 19c are similar to those of the cell immobilization device 19b of the third Embodiment, therefore, the explanation thereof is now omitted.

### Fifth Embodiment

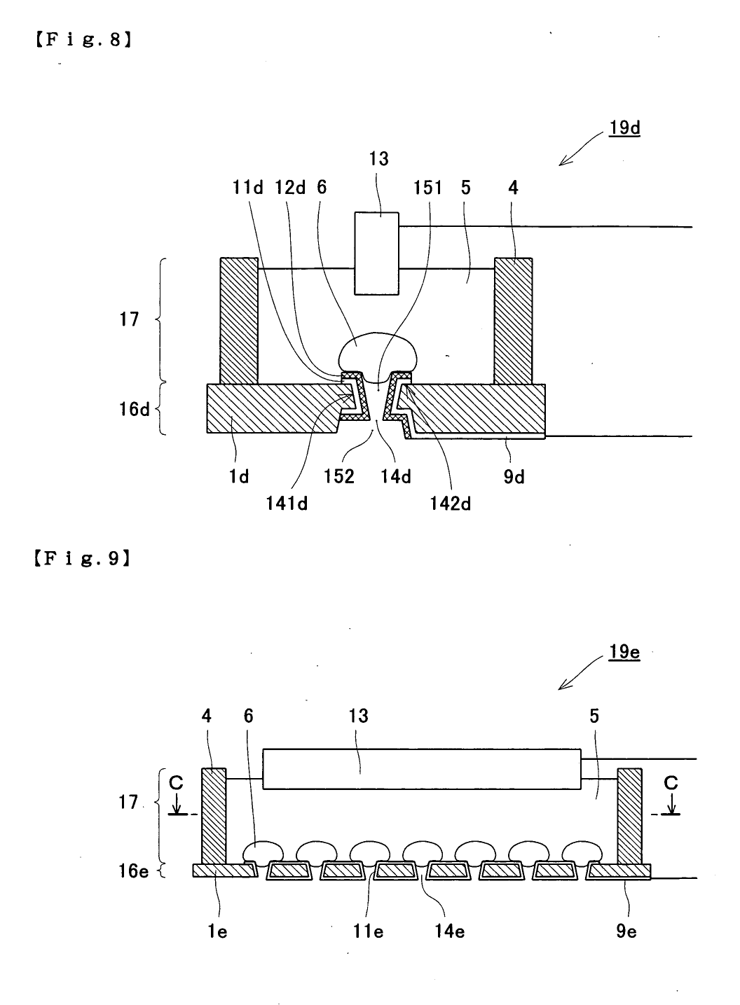

FIG. 8 is a cross sectional drawing schematically showing the constitution of the cell immobilization device 19d of this Embodiment. Because the cell immobilization device 19d has a different constitution in only the sensor part from the cell immobilization device 19 of the first Embodiment, the explanation of other constitutions except for the sensor part is omitted through assigning the identical number.

A substrate 1d has a through-hole 14d. An electrode 11d is formed on the hole wall surface 141d and the marginal edge of the hole opening 142d of the through-hole 14d. The electrode 11d is formed by making an electrode material adhered on the hole wall surface 141d and the marginal edge of the opening 142d of the through-hole 14d using a vacuum vapor deposition method or a sputtering method. The surface of the electrode 11d is coated with a dielectric layer 12d. For coating of the electrode 11d with the dielectric layer 12d, a similar method to that in the first Embodiment can be employed. Coating of the electrode 11d with the dielectric layer 12d may be carried out such that the capacitance of the electric double layer of the interface with a 0.1 M electrolyte solution of the electrode 11d becomes preferably equal to or greater than 25 μF/cm², more preferably equal to or greater than 27 μF/cm², with the applied voltage of 0 V, similarly to the first Embodiment.

On the lower face of the sensor part 16d is formed a lead wire 9d such that it connects to the electrode 11d. The lead wire 9d may be constituted such that it is formed on the upper face of the sensor part 16d.

The cell immobilization device l9d of this Embodiment differs from the cell immobilization device 19b of the third Embodiment only in the shape of the through-hole formed in the sensor part. The through-hole 14d is formed by integrating a truncated cone-shaped recession 151 formed from the upper face of the substrate 1d, and a truncated cone-shaped recession 152 formed from the lower face of the substrate 1d. The through-hole 14d can be readily formed by such a method of formation even though production of a continuous truncated cone-shaped through-hole is difficult owing to the thickness of the substrate 1d.

### Sixth Embodiment

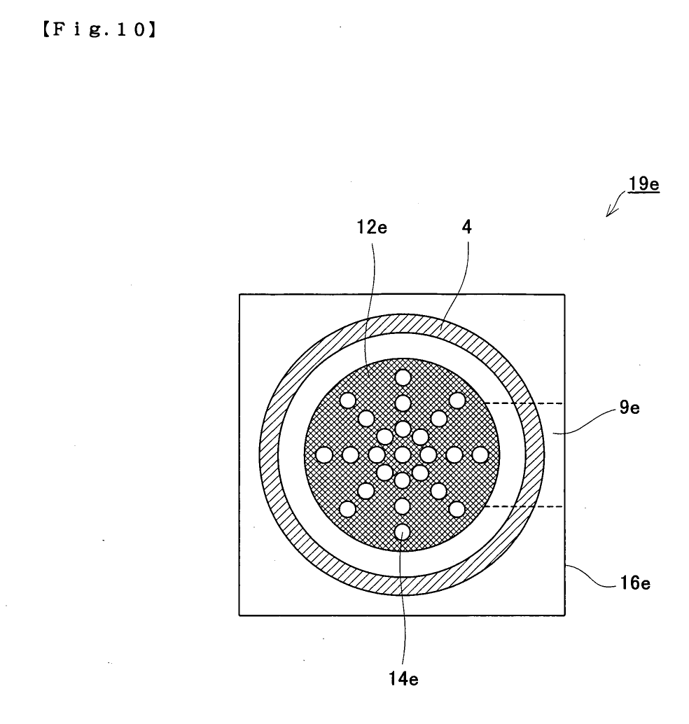

FIG. 9 is a cross sectional drawing schematically showing the constitution of the cell immobilization device 19e of this Embodiment. FIG. 10 is a cross sectional drawing viewed along the line C-C depicted in FIG. 9. However, FIG. 9 illustrates the state in which a cell 6 is immobilized in the cell immobilization device 19e, but FIG. 10 illustrates with the cell 6 omitted therefrom. Moreover, in FIG. 10, a lead wire 9e formed on the lower face of the sensor part 16 is illustrated by a dashed line. Because the cell immobilization device 19e has a different constitution in only the sensor part from the cell immobilization device 19 of the first Embodiment, the explanation of other constitutions except for the sensor part is omitted through assigning the identical number.

The sensor part 16e has a constitution in which multiple number of through-holes 14e having a similar shape to the through-hole 14b shown in FIG. 5 are formed per one electrode 11e. As is shown in FIG. 10 (not shown in FIG. 9) , the electrode 11e is coated with a dielectric layer 12e. The through-holes 14e are formed, 25 in number, in a radial pattern on a substrate 1e. However, number and positional relation ship of the through-holes 14e are not limited thereto. In this Embodiment, electrical signals resulting from cells 6 retained in multiple through-holes 14e has come to be detected by one electrode 11e as one electrical signal. The cell immobilization device 19e of this Embodiment is useful in screening of a drug and the like in which responsiveness of multiple cells is concomitantly detected.

### Seventh Embodiment

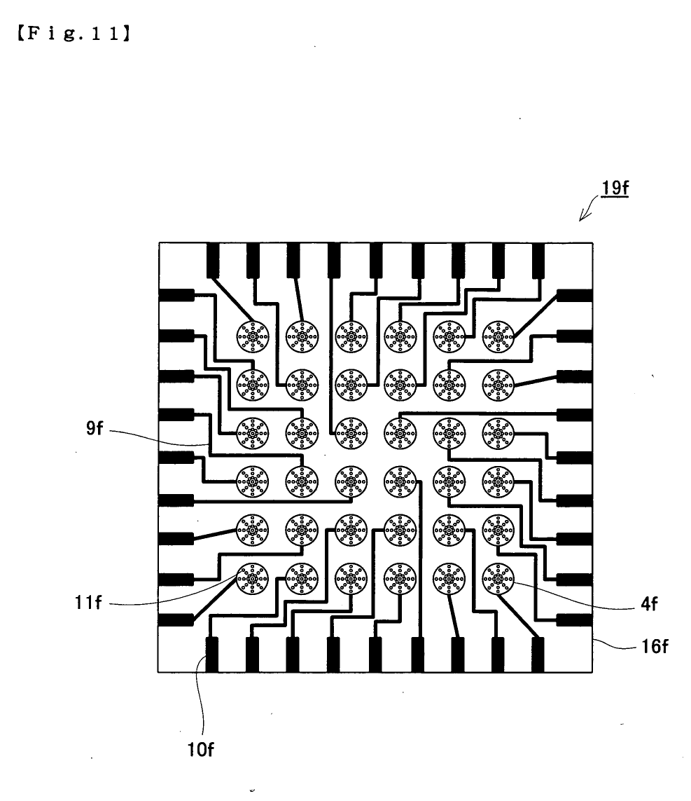

FIG. 11 is a top view showing the sensor part 16f of the cell immobilization device 19f according to the seventh Embodiment. A lead wire 9f is not appeared on the upper face because it is formed on the lower face of the sensor part 16f, however, in FIG. 11, the lead wire 9f is schematically depicted. The cell immobilization device 19f is constituted such that electrodes 11f are arranged at each intersecting point of a lattice having 6 rows and 6 columns. The cell immobilization device 19e of the sixth Embodiment has the constitution having only one electrode 11e formed on the sensor part 16e, however, the cell immobilization device 19f of this Embodiment is constituted to have multiple electrodes 11f and corresponding lead wires 9f formed on the sensor part 16f.

Although FIG. 11 illustrates with a solution retaining part omitted therefrom, the divider member that constitutes the solution retaining part may be provided either per every one electrode 11f, or per a set of multiple electrodes 11f. The constitution provided with the divider member per every one electrode 11f is useful, for example, in the measurement of responsiveness to a drug of the immobilized cell on each electrode 11f, whilst the constitution provided with the divider member per a set of multiple electrodes 11f is useful, for example, in carrying out the analysis of a network because a network can be formed among nerve cells immobilized on each electrode 11f. A lead wire 9f is not formed on the upper face of the sensor part 16f, therefore, the sensor part 16f and the divider member are not necessarily constituted separately, but it is possible to give an integrated formation.

Other constitutions, methods of use and the like of the cell immobilization device 19f are similar to those of the cell immobilization device 19e of the sixth Embodiment, therefore, the explanation thereof is now omitted.

The present invention is more specifically explained below by way of Examples. These Examples do not limit the present invention.

### EXAMPLE 1

The cell immobilization device 19e according to the sixth Embodiment (with different number of through-holes 14e from that shown in FIG. 10) (: Example 1), and a cell immobilization device which is different from the cell immobilization device 19e of Example 1 only in such a respect that the electrode 11e is not coated with a dielectric layer 12e (: Comparative Example 1) were produced, and subjected to experiments for determining the characteristics of the electrode.

As a substrate 1, a PET film which is a square with 100 mm sides and the thickness of 25 μm was used, and a material for the electrode 11e and the lead wire 9e, gold was used. On the PET film were formed through-holes 14e using a pulse oscillated excimer laser. The through-holes 14e were formed in multiple regions, 100 in number per each region. In each region, the 100 through-holes 14e were formed to give a radial pattern. Each through-hole 14e had a truncated cone shape with the diameter of the opening on the upper face of 20 μm, and the diameter of the opening on the lower face of 5 μm. One region was within a size included in the range of the diameter of 1 mm. The spaces between each region were set to be: horizontal interval of 3 mm, and longitudinal interval of 10 mm between adjacent regions in order to facilitate the cutting out into small pieces in the following step.

Then, gold which is an electrode material was subjected to sputtering such that the thickness became 20 nm, through a stencil mask which had been formed in such a manner that each hole, which was slightly larger than the opening of the through-hole 14e on the upper face of the substrate 1e, corresponded to each through-hole 14e. Then, the shape of the lead wire 9e was sputtered on the lower face of the substrate 1e by a similar method as described above. The electrode 11e formed within the area of the circle with the diameter of 1 mm on the upper face of the substrate 1e and on the inside wall of the through-hole 14e was thereby connected to the lead wire 9e formed on the back face. Thereafter, the PET film which was a square with 100 mm sides was cut into multiple small pieces such that each one small piece includes one region. The small piece which was cut out was ascertained for continuity starting from the upper face of the electrode 11e formed on the upper face of the film reaching to the lead wire 9e. One small piece was defined as a sensor part of Comparative Example 1.

In Example 1, the following treatment was further conducted. A dielectric material solution including PEI diluted in borate buffer, pH 8.4, to give the final concentration of 0.1% by weight was exposed on the sensor part for 16 hours, and thereafter the dielectric material solution was removed. Then, the surface of the sensor part was sufficiently rinsed with sterile water. Accordingly, a sensor part of Example 1 was formed having a dielectric layer 12e formed on the upper face of the electrode 11e.

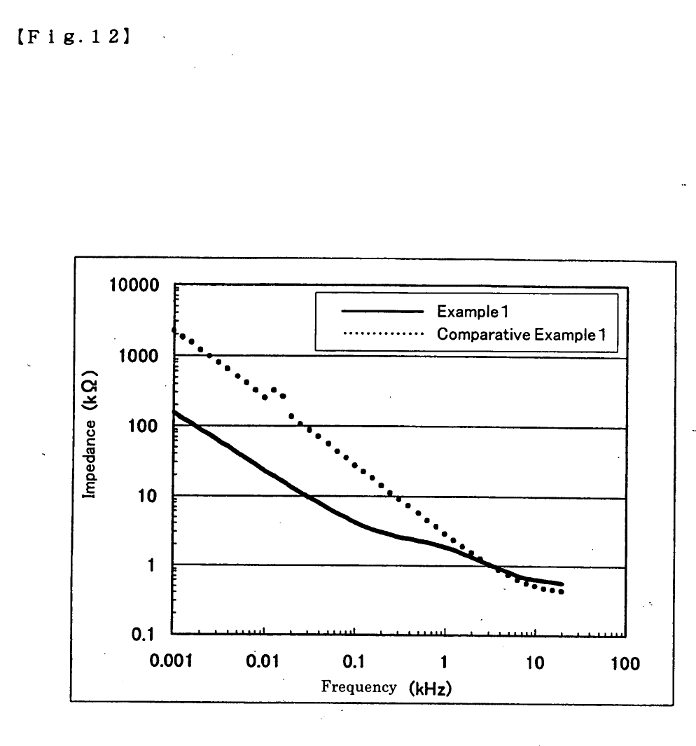

Cell immobilization devices were respectively produced using the sensor parts of Example 1 and Comparative Example 1, and the impedance of the circuit including the cell immobilization device was measured. For measurement of the impedance, a platinum electrode was used as a standard electrode, and 0.1 M aqueous sodium chloride solution was filled in the solution retaining part. Using the electrode on the sensor part in Example 1 and Comparative Example 1 as an electrode for the measurement, and using an electrochemical measurement system HC3000 (manufactured by HOKUTO DENKO Co., Ltd.), impedance of the circuit was measured with continuously varying the frequency of from 1 Hz to 20 kHz, while applying a sinusoidal voltage between the electrodes. Bias voltage employed was set to be 0 V, and amplitude of the applied voltage was set to be 50 mV. FIG. 12 shows the measurement results. As is shown in FIG. 12, marked differences are found between the impedance of Example 1 and Comparative Example 1. When the frequency is 3 kHz or less, the impedance in Example 1 is less than the impedance in Comparative Example 1. Such a tendency was more remarkable as the applied frequency is lower. In the range of lower frequency of 20 Hz or less was applied, thus resulting difference of the impedance was tenth or lower.

In this measurement, the impedance is composite impedance which includes capacitance formed in the interface between the measurement electrode and the solution, and in the interface between the standard electrode and the solution, solution resistance, electrode surface resistance, circuit resistance, and the like. However, from the measurement results shown in FIG. 12, it is revealed that the difference of impedance in Example 1 and Comparative Example 1 is dependent on frequency. Therefore, the difference of both impedance is found to result from frequency dependent capacitance formed in the interface between the measurement electrode and the solution. Although the impedance resulting from the capacitance formed in the interface between the standard electrode and the solution is also dependent on frequency, there exists any difference between the constitutions of both standard electrodes, therefore, it is believed that such impedance is not responsible for the difference of impedance demonstrated by the measurement results shown in FIG. 12.

Accordingly, it can be concluded that the comparison between the impedance in Example 1 and the impedance in Comparative Example 1 in the range of low frequency is equivalent to the comparison of the impedance of capacitance in the interface of the measurement electrode. Thus, it can be concluded that the impedance of capacitance in the interface of the measurement electrode in Example 1 is tenth or lower compared to that in Comparative Example 1, in the range of low frequency.

When the capacitance of the electric double layer of the interface of the measurement electrodes of Example 1 and Comparative Example 1 was calculated from the measurement results of the impedance as described above, a value of 165 μF/cm² was derived in Example 1, and a value of 10 μF/cm² was derived in Comparative Example 1. Capacitance of the electric double layer may vary depending on the applied voltage and the concentration of electrolytes, however, the values calculated above were derived with the applied voltage of 0 V, and the electrode concentration of 0.1 M.

### EXAMPLE 2

Sensor parts that are similar to those in Example 1 and Comparative Example 1 were produced, and on each sensor part including the electrode surface was coated collagen (Sigma P-4511), which is a cell adhesive protein, as an immobilization material for one hour by a predetermined method (in an atmosphere at 37° C.). The sensor parts produced in such a manner were referred to as sensor parts of Example 2 (with PEI coating) and Comparative Example 2 (without PEI coating).

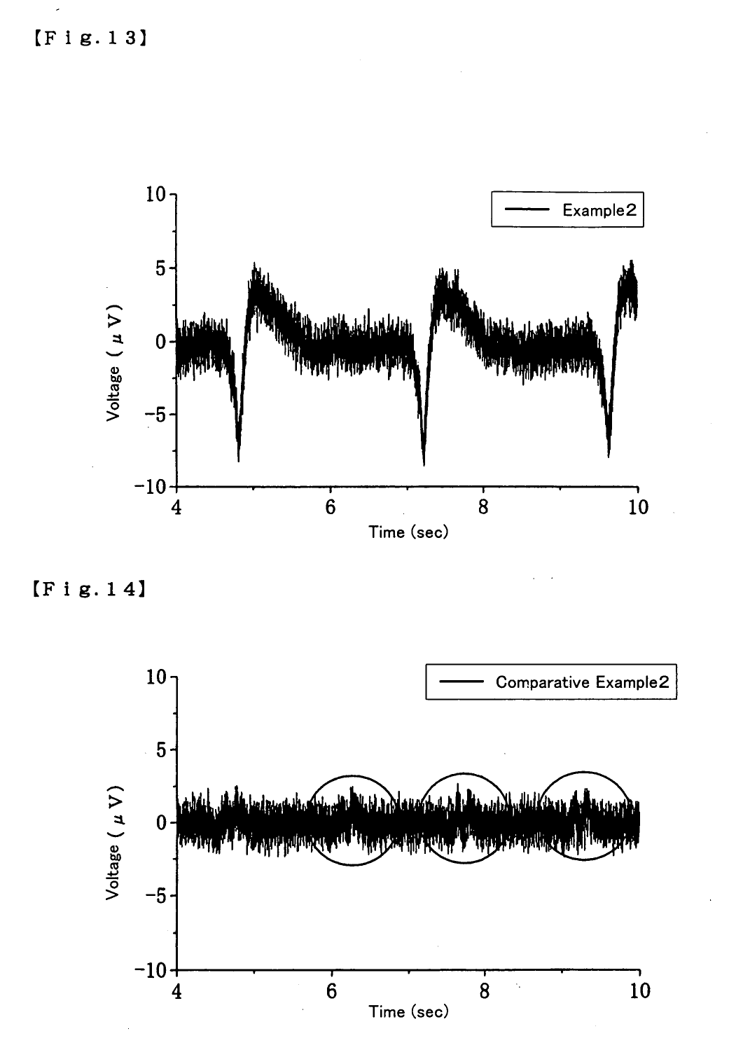

A divider member made of acryl was adhered on each sensor part to form a solution retaining region, and thereafter, the solution retaining region was filled with a culture medium. Then, a smooth muscle cell derived from rat aorta VSMCs A-10 (ATCC CRL-1476) was seeded such that the density became 2×10⁴ cells/ml, and cultured in a CO₂ incubator for 5 days (in an atmosphere at 37° C., 5% CO₂). The culture medium employed was DMEM+10% FBS. Voltages which were measured using the sensor part of Example 2 and Comparative Example 2 (voltage between the standard electrode and the measurement electrode) are shown in FIG. 13 and FIG. 14, respectively. The horizontal axis indicates the time, and the longitudinal axis indicates the voltage (i.e., intensity of the electric potential exhibiting the activity of the cell). Periodic activity of the cell detected in FIG. 13 is scarcely detected in FIG. 14. Also in FIG. 14, a periodic change is detected in the site enclosed with a circle, however, the change of the electrical signal is small in comparison with FIG. 13.

### EXAMPLE 3

Sensor parts that are similar to those in Example 1 and Comparative Example 1 were produced, which were referred to as sensor parts of Example 3 (with PEI coating) and Comparative Example 3 (without PEI coating).

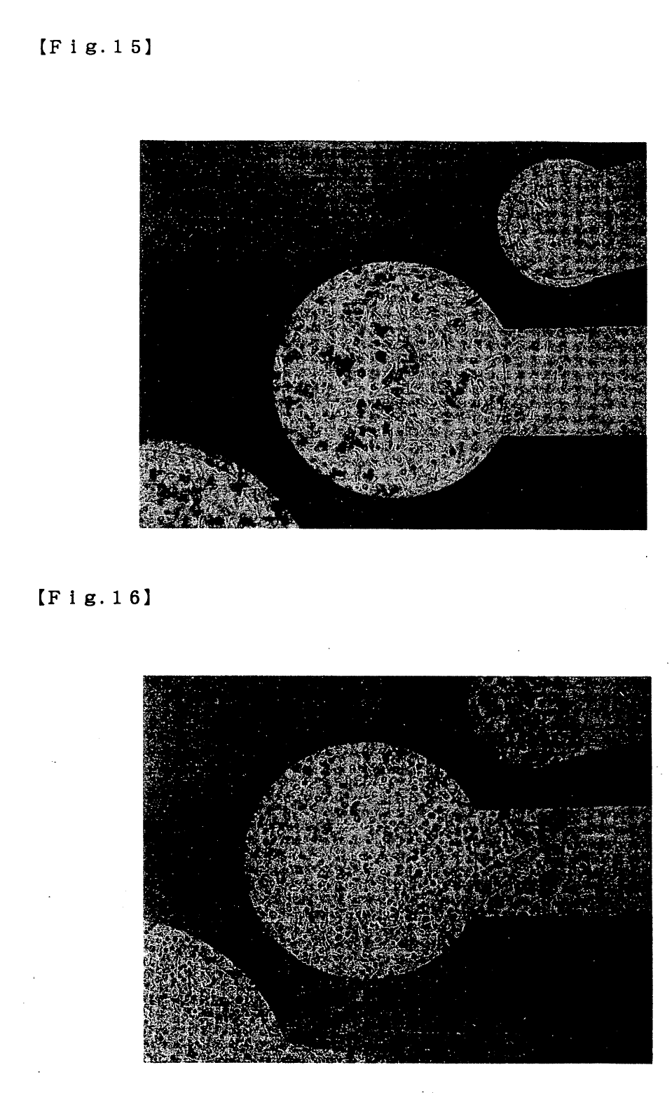

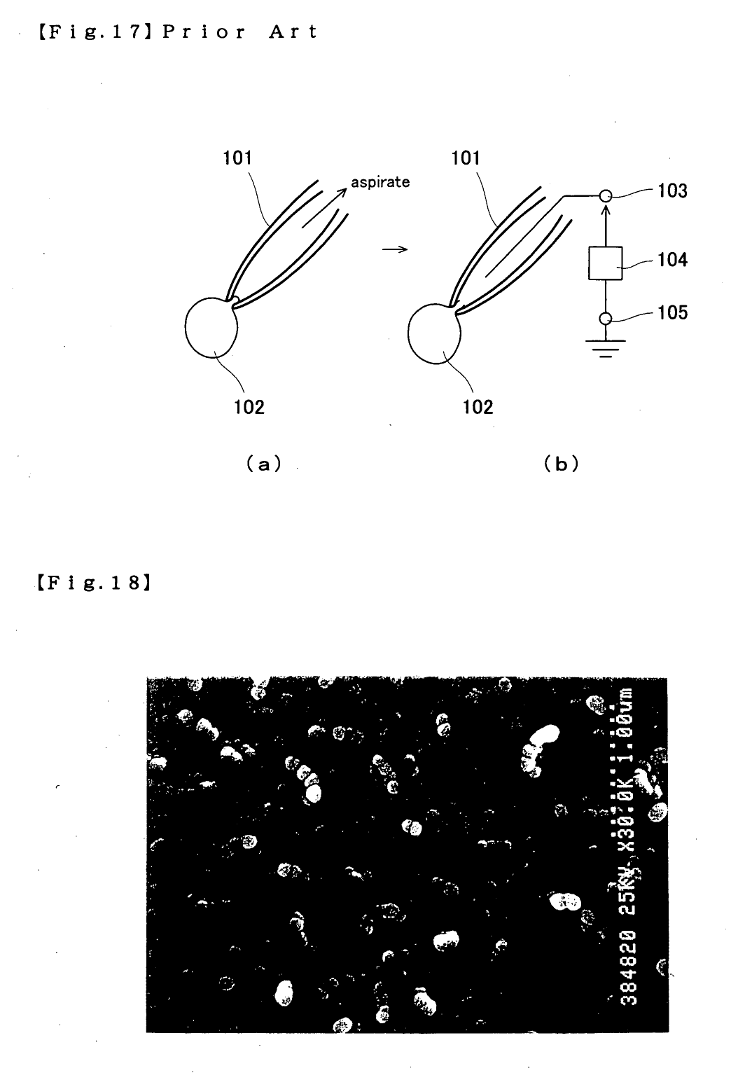

A divider member made of acryl was adhered on each sensor part to form a solution retaining region, and thereafter, the solution retaining region was filled with a culture medium. Then, a smooth muscle cell derived from rat aorta A7r5 was seeded such that the density became 1×10⁶ cells/ml, and cultured in a CO₂ incubator (in an atmosphere at 37° C., 5% CO₂). The culture medium employed was DMEM+10% FBS. Stereoscopic microscopy photographs of the upper face of the sensor part after the culture for 2 days in Example 3 and Comparative Example 3 are respectively shown in FIG. 15 and FIG. 16. The smooth muscle cell derived from rat aorta A7r5 becomes spherical shape upon isolation, under the pressure from the solution in an isotropic manner. Then, after the settlement on the substrate, the aforementioned cell transforms into a spindle shape through extending the cytoskeleton within the cell. As is clear from the comparison of stereoscopic microscopy photographs shown in FIG. 15 and FIG. 16, settlement of the cells proceeded in Example 3, however, the cells scarcely settled in Comparative Example 3. In summary, it is revealed that a difference in settling property of a cell on the upper face of the electrode is caused depending on whether or not the upper face of the electrode on the sensor substrate is coated with PEI.

From the description hereinabove, many modifications and other embodiments will be apparent to persons skilled in the art. Therefore, the above description should be construed as merely illustrative exemplification, which is provided for the purpose of teaching the best embodiment for carrying out the present invention. Details of the structure and/or function thereof can be substantially altered without departing from the spirit of the present invention.

What is claimed is:

1. An integrated electrode comprising a substrate equipped with at least one electric conductor, and a wiring part which leads an electrical signal out from said electric conductor,
said integrated electrode can detect an electrical signal resulting from an electrophysiological change of a cell immobilized on the surface of said electric conductor,
wherein at least a part of the surface of said electric conductor is coated with a dielectric material,
said dielectric material is a positively charged polymer material, and
said cell is an isolated cell and/or a cultured cell.

2. The integrated electrode according to claim 1 wherein said integrated electrode is constituted such that said cell is immobilized on the surface of said electric conductor without the degeneration of the cell.

3. The integrated electrode according to claim 1 wherein said dielectric material comprises a polymer material selected from the group consisting of polyethyleneimine, polyornithine, and polylysine.

4. The integrated electrode according to claim 1 wherein said dielectric material comprises a polymer material having a biguanide group or a carbamoylguanide group.

5. The integrated electrode according to claim 1 wherein said electric conductor comprises a material which includes a material selected from the group consisting of platinum, gold, palladium, rhodium, silver, tungsten, ITO, and any mixtures thereof.

6. The integrated electrode according to claim 1 wherein at least a part of the surface of said electric conductor coated with said dielectric material is further coated with an immobilization material,
said immobilization material is a material that provides an electrostatic interaction and/or intermolecular force with said cell, and that is different from said dielectric material.

7. The integrated electrode according to claim 6 wherein said immobilization material is a cell adhesive protein.

8. The integrated electrode according to claim 1 wherein the capacitance of the electric double layer of the interface with a 0.1 M electrolyte solution of said electric conductor is 27 μF/cm² or greater at the applied voltage of 0 V.

9. The integrated electrode according to claim 1 wherein said electric conductor is formed within at least one through-hole formed to the substrate and/or around said through-hole on the upper face of said substrate.

10. The integrated electrode according to claim 9 wherein one said electric conductor is formed per one said through-hole.

11. The integrated electrode according to claim 9 wherein one said electric conductor is formed per a set of multiple said through-holes.

12. A cell immobilization device which is equipped with the integrated electrode according to claim 1, and a solution retaining part for culturing said cell in a region including the surface of said electric conductor of said integrated electrode.

13. The cell immobilization device according to claim 12 wherein said integrated electrode is equipped with multiple said electric conductors, and said solution retaining part is separated per each one electric conductor.

14. The cell immobilization device according to claim 12 wherein said integrated electrode is equipped with multiple said electric conductors, and said solution retaining part is separated per a set of said multiple electric conductors.
